(19) United States
(12) Patent Application Publication
(10) Pub. No.: US 2019/0015667 A1
(43) Pub. Date: Jan. 17, 2019

(54) HUB-BASED STRATEGY FOR REDUCING HARDWARE AND ALGORITHMIC SUPPORT NEEDS IN A LEAD-LESS PACING SYSTEM THAT LEVERAGES TRIGGERED MESSAGING THROUGH A BODY AREA NETWORK

(71) Applicant: BIOTRONIK SE & Co. KG, Berlin (DE)

(72) Inventors: Brian M. Taff, Portland, OR (US); Jeffrey A. von Arx, Lake Oswego, OR (US); Hannes Kraetschmer, West Linn, OR (US); Larry Stotts, Tigard, OR (US)

(21) Appl. No.: 16/019,755

(22) Filed: Jun. 27, 2018

Related U.S. Application Data
(60) Provisional application No. 62/530,863, filed on Jul. 11, 2017.

(51) Int. Cl. A61N 1/37; A61B 5/042; A61N 1/39; A61B 5/00; H04W 4/38

(52) U.S. Cl. CPC A61N 1/3702; A61B 5/042; H04W 4/38; A61B 5/0006; A61N 1/3956

(57) ABSTRACT

An implantable device system for applying electrical stimulation to a patient, including: a first implantable device configured to measure at least one parameter indicative of a physiological or an activity state of the patient, a second implantable device configured to apply and/or adapt electrical stimulation to the patient in response to said at least one parameter, and wherein the first implantable device is further configured to communicate information pertaining to said at least one parameter to the second implantable device.

### CROSS-REFERENCE TO RELATED APPLICATIONS

This patent application claims the benefit of co-pending U.S. Provisional Patent Application No. 62/530,863, filed on Jul. 11, 2017, which is hereby incorporated by reference in its entirety.

### TECHNICAL FIELD

The present invention is directed to implantable device systems and, more particularly, to implantable device systems for applying electrical stimulation to a patient in response to at least one measured parameter indicative of a physiological or an activity state of the patient.

### BACKGROUND

Rate adaptation, capture control, holter management, and remote monitoring support represent baseline pacemaker features in state of the art pacemakers. To date, their implementation in implantable lead-less pacers (LP) has either been absent or relied upon the packing of relevant hardware (inclusive of memory allocations) and circuitry support directly into the LP.

Additionally, the LP itself owns the algorithmic responsibility for managing input gathered from the feature support hardware to adapt, when appropriate, to prevailing, monitored conditions. The therapy implant, which is heavily constrained from both size (needing to reside within the heart and enable routing through the vasculature during implantation) and power support (needing to provide service lifetimes comparable to leaded pacers, but in a much smaller volume), thereby carries the full burden associated with efforts to enable baseline feature management.

In the specific case of remote monitoring, the in-body depth of lead-less pacemakers (typically they reside 5 to 15 cm beneath the skin) confounds the ability for the implants to communicate with standard wireless remote monitoring infrastructures, and the physically small battery makes it infeasible to transmit from a lead-less pacemaker with a relatively high power RF far-field transmitter. To remedy this, a patient may need to apply a wand over the device and extract data via direct interactions leveraging near-proximity coil-based communication methods. However, such efforts, while useful for enabling data collection, demand a level of patient compliance for remote monitoring support that surpasses that associated with standard electro-active cardiac care devices.

Particularly, U.S. Publication No. 2013/0027186 discloses a pacemaker that communicates via a communication link with an external device.

Based on the above, a problem to be solved by the present invention is to provide an implantable device system that is improved concerning one or several of the above-described difficulties.

The present invention is directed at overcoming one or more of the above-mentioned problems.

### SUMMARY

At least the above problem is solved by an implantable device system having the features of claim 1. Embodiments of the present invention are stated in the sub claims and are described below.

According to claim 1 an implantable device system for applying electrical stimulation to a patient is disclosed, the system comprising:
a first implantable device configured to measure at least one parameter indicative of a physiological state of the patient (e.g. the condition or state of the body or bodily functions of the patient) and/or of an activity state of the patient when the first implantable device is implanted into the body of the patient,
a second implantable device configured to apply and/or adapt electrical stimulation to the patient in response to said at least one parameter for therapy purposes when the second implantable device is implanted into the body of said patient, and
wherein the first implantable device is further configured to communicate said at least one parameter to the second implantable device.

The respective physiological state can be measured by suitable sensors and methods which are known in the state of the art. The physiological state can be measured by means of parameters such as: parameters related to the electrocardiogram of the patient, Q-T-Interval, characteristics of QRS depolarization, ST segment elevation, cardiac contractility, temperature of the patient, minute ventilation of the patient, etc. Furthermore, the activity state may be sensed by motion sensors such as, for example, an accelerometer or a vibration sensor, wherein the vibration sensor is for instance implemented as piezo sensing element or a capacitive sensing element. Correspondingly, the measured acceleration of the body of the patient or the output signals measured with these sensors may serve as measures for deriving the activity state.

Particularly, in case the second implantable device is an LP, the present invention overcomes the usual system limitations of classical LP designs by enabling wireless data transmissions, as for instance via RF, from the first implantable device (that particularly forms a cardiac monitoring hub) that is stationed at a much shallower subcutaneous in-body depth (typically ˜0.5 to 3 cm beneath the skin) and can relay information descriptive of the deeper implant's (i.e., LP's) conditions.

According to an embodiment of the present inventive implantable device system, the device system comprises besides the first implantable device, more devices configured to measure at least one parameter indicative of a physiological state of the patient. For instance, the device system may comprise several LPs which are implanted at different locations of a heart, for example, one in the right atrium and one in the right ventricle, whereas the LPs are configured to communicate data to the second device.

Moreover, the first implantable device is configured to transmit data to an external unit via wireless data transmission, as for instance via RF. That external unit may be an external device, as for example a programmer device for remote/wireless programming of the first and/or the second implantable device; the external unit may be a patient device where the data may be further processed, evaluated, displayed to users or the patient, sent to other devices or a service center, or the like, for the purpose of remote monitoring of the physiological state of the patient. In an embodiment, the external unit may be an external service center where the data may be further processed, evaluated or the like, for the purpose of remote monitoring of the physiological state of the patient.

The present invention allows reducing direct algorithmic and hardware support needs in LPs residing within the heart volume and repartitions much of that responsibility to said peripheral subcutaneous cardiac monitoring hub (first implantable device) enabled with state change triggering support. The second implantable device according to the present invention, as e.g., LPs typically have strict requirements regarding a small device size, therefore the volume for accommodating hardware and the battery is strongly limited. It is not possible to implement complex hardware and/or batteries inside the second device. According to the present invention, device components, as e.g., hardware for wireless data transmission or sensors are implemented in the first device (the hub device), instead of inside the second device, which allows reducing device volume and power consumption of the second device, leading to an extended battery lifetime.

Thus, particularly, the present invention removes dedicated components, circuitry, and algorithmic support from the second implantable device (e.g., LP) and stations it instead within the first implantable device (e.g., in said in-body cardiac monitoring hub) to enable a revised approach to (lead-less) pacemaker feature support. Particularly, this strategy leans on maturing efforts to align implanted medical devices with body area network configurations capable of expanded/improved patient care. In this approach, said hub likely resides in a subcutaneous pocket and may embody in certain embodiments either a monitoring-focused implementation or an implant also capable of delivering therapeutic output, e.g., a subcutaneous implantable cardioverter-defibrillator (s-ICD) configured to apply electrical stimulation to the heart for providing defibrillation of the heart of the patient.

In the embodiment where the first device is a subcutaneous hub and the second device is LP residing inside the heart, the first device is much more easily replaced due to its shallow implant location, whereas the second device may not be. This means that the battery longevity of the first device can be in the range of 6 yrs, whereas the battery longevity of the second device needs to likely be 10 years or more.

Particularly, according to an embodiment of the present invention, the second implantable device is a pacemaker configured to apply electrical stimulation to a heart of the patient.

Particularly, according to an embodiment, the second implantable device is an implantable lead-less pacemaker (LP) that is configured to be implanted into a chamber (right ventricle, left ventricle, right atrium or left atrium) or on a chamber (e.g., epicardially on the outside of the right ventricle or of the left ventricle) of said heart of the patient (e.g., via a catheter). The LP may comprise a hermetically sealed housing enclosing a pulse generator for generating said pacing pulses and a battery for supplying energy to the pulse generator. The lead-less pacemaker may further comprise fastening means provided on the housing for fastening the lead-less pacemaker to the chamber (ventricle or atrium), and a pacing electrode (e.g., a cathode and an anode) provided, e.g., on a face side of the housing of the lead-less pacemaker for applying the electrical stimulation to the heart. Regarding the LP, the notion lead-less means that the electrode of the LP is not connected via an external lead to the housing of the pacemaker, but is provided on the housing. Such a lead-less pacemaker is for instance disclosed in U.S. Pat. No. 9,358,387 which is incorporated herein in its entirety by reference. Note that some lead-less pacemakers incorporate a lead extension which can allow sensing or stimulation of a different chamber of the heart than the main body of the LP resides in. Such devices are also included in the notion of lead-less pacemakers.

In one embodiment, there are two or more second implantable devices (20) in communication with one hub (10). For example, there may be a LP (20) in the RV, another in the RA, and another attached to the LV (either in the LV or located epicardially). One, two, or all three of these LP devices may be in communication with a single hub (10).

Particularly, according to an embodiment of the present invention, the second implantable device (e.g., LP) is configured to apply electrical stimulation to the patient (e.g., to the patient's heart) in the form of electrical pulses having a pulse rate, wherein the device system is configured to adapt said electrical stimulation by adapting said pulse rate, and wherein the first implantable device (e.g., hub) is configured to measure said at least one parameter indicative of an activity (or a physiological) state of the patient, wherein the first implantable device is further configured to communicate a signal (e.g., targeted in-body messaging) to the second implantable device in case said activity state exceeds one or more pre-defined thresholds, wherein in turn the second implantable device (e.g., the LP) is configured to adapt the pulse rate in response to said signal.

Assuming, e.g., an accelerometer-based rate adaptation scheme (here a measure for the activity state in an acceleration as, e.g., measured with accelerometer), the first implantable device (e.g., hub) would remove such hardware from the second implantable device (pacemaker, particularly LP) along with any power, control, and algorithmic decision-making infrastructure needed for its management. Particularly, said accelerometer is therefore arranged in the first implantable device, e.g., in said hub.

Further, by assessing activity at the first implantable device, the system could evaluate the need for rate adjustments without needing to winnow such information from a sensing stream influenced by a surrounding local motion micro-environment. In contrast, in a more conventional arrangement when the accelerometer is located within the 2nd device (the LP) the measured accelerations come from activity (signal of interest) and cardiac motion (signal not of interest). The improved signal-to-noise configuration that comes from having the activity sensing accelerometer located outside of the beating heart would likely provide improved reliability for any system-dependent rate adaptation efforts in terms of its correspondence with true patient demand.

For capture control purposes, the first implantable device (e.g., hub) would likely reside in a better anatomical position for capturing overarching cardiac electrical behaviors using vectors more closely aligned with those akin to surface ECGs. The first implantable device (e.g., hub) could periodically assess the correspondence of sensed cardiac responses to known lead-less pacing output and grade its efficacy.

Particularly, in an embodiment of the present invention, the first implantable device (e.g., hub) is configured to sense cardiac responses of the patient (e.g., in the form of an ECG) to the known electrical stimulation of the second implantable device (e.g., LP), wherein the first implantable device is configured to periodically assess the correspondence of said sensed cardiac responses of the patient to said electrical stimulation for grading its efficacy (e.g., for enabling myocardial capture via template morphology comparisons or otherwise).

This system configuration would additionally support a new approach to holter management via triggered streams. Rather than forcing the second implantable device (e.g., lead-less pacer) to make memory accommodations for supporting such a feature, an interaction between the second and the first implantable device could occur where first implantable device (e.g., the hub) or the second implantable device (e.g., LP) could recognize a condition worthy of holter storage, and a triggered message could transition the first and second implantable devices (e.g., pacer and hub) into a dialogue mode where the second implantable device (e.g., LP) streams its locally sensed data to the first implantable device (e.g., hub) for remote storage.

Particularly, according to an embodiment of the present invention, the first implantable device (e.g., said hub) is therefore configured to trigger a signal when the at least one measured parameter (or another parameter measured by the first implantable device) meets a pre-defined criterion (indicating said holter storage is need), wherein the signal upon reception by the second implantable device transitions the first and the second implantable device into a dialogue mode, where the second implantable device (e.g., LP) streams data (e.g., indicative of a condition of the heart of the patient) sensed by the second implantable device to the first implantable device (e.g., hub) for remote storage. Particularly, said data corresponds to an electrocardiogram (ECG) of the patient's heart.

Such a setup would mean that information sensed within the heart could be retained in the less volumetrically constrained first implantable device (e.g., hub). Further, coordinated snapshots would prove accessible, wherein the first implantable device captures a sensed patient response of its own while also collecting the sensed data from the in-heart pacer. By collecting two electrical signatures of patient behaviors assessed from different vantages within a shared temporal survey window, it may prove possible to improve upon meaningful clinical diagnostic capabilities. Thus, according to an embodiment, the first implantable device particularly comprises the function of a cardiac monitoring hub (see also below).

Particularly, according to a further embodiment, the first implantable device (e.g., hub) may be configured to conduct all remote monitoring interactions and even constitute the only device of the system that is capable of supporting direct interactions with an outside programmer (i.e., as a conduit for programming the second implantable device (e.g., LP), and for interrogating the second implantable device (e.g., LP), and further for performing follow-up tests on the second implantable device (e.g., LP)). Thus, due to this system architecture, inductive coils are removed from all of the non-hub implants (i.e., from all implants but the first implantable device). In other words, with the present invention, all in-body communication merely uses non-coil-based means according to an embodiment and the first implantable device (e.g., hub) provisions both RF interactions with a standard wireless remote monitoring infrastructure and RF or coil-based interactions with an external programmer according to an embodiment of the present invention. This systematic design strategy would thus enable an on-ramp for remote monitoring support in lead-less pacers that pairs with the standard wireless relay infrastructures and, at least for first implantable devices (e.g., hubs) incorporating Bluetooth (including BLE [Bluetooth Low Energy]) support, serve as a framework for enabling patient interactions with lead-less pacers via connected health, which is an emerging approach to enabling patient access and annotation support to biometrics emergent from medical devices and may employ a Bluetooth communication infrastructure and pairings with patient-owned smart devices.

In one embodiment, changes in patient metabolic demand, capture control support, triggered holter management, and remote monitoring support may not mandate beat-by-beat interactions. In this case the data exchange between the first implantable device (e.g., hub) and the second implantable device (e.g., LP) may exist on an as-needed or periodic basis. Such configurations provide allowance for message retry support without detrimentally impacting the intended therapies. Upon receipt of state change request information, the deeper implanted second implantable device (e.g., LP) could adapt rate, therapy output, sensor streaming support, and/or relay locally retained statistics to realize improved patient care. In another embodiment, the periodic basis may include beat-by-beat interactions, e.g., if clinical benefit exists.

Particularly, according to an embodiment of the present invention, the first implantable device is an implantable loop recorder, i.e., it is configured (among other possible functionalities) to receive electrocardiogram signals from the heart of the patient and is further configured to store said signals in a memory, wherein said memory particularly is a circular memory that gets overwritten after having stored a pre-defined amount of data.

In a further embodiment of this invention, some patients are able to benefit from a synchronous pacing mode which is a challenging demand for a single chamber lead-less pacemaker wholly residing within the patient's ventricle. In patients where the first cardiac device (the implantable loop recorder) is able to detect a p-wave, it can communicate this to the second cardiac device (the LP) and synchronous pacing therapy can be given. Although the p-wave is difficult to reliably detect with implantable monitors in all patients all the time, in many patients it can be detected a least part of the time, and in such patients synchronous pacing can be achieved at least part of the time with this invention.

According to an embodiment of the present invention, the first implantable device comprises at least one sensor for measuring at least one parameter indicating a physiological state of the patient. That sensor may be for example an acceleration sensor or a temperature sensor.

Alternatively, the first implantable device may further form a subcutaneous implantable cardioverter-defibrillator (s-ICD) that is configured to apply electrical stimulation (particularly shock) to the heart for providing defibrillation of the heart.

In one embodiment, the first and the second implantable device are configured to communicate via modulated e-field signals (small current placed between electrodes in the body). In a second embodiment, the first and second implantable devices are configured to communicate via modulated ultrasonic signals. These methods of communication are preferred because they propagate well in the body and required orders of magnitude less energy than many alternatives.

According to an embodiment of the present invention, a method for operating a device system is proposed, comprising the steps:
measuring at least one parameter indicative of a physiological or an activity state of the patient via a first implantable device,
communicating information pertaining to said at least one parameter from the first implantable device to a second implantable device,
applying and/or adapting electrical stimulation to the patient in response to said at least one parameter via the second implantable device.

According to an embodiment of the method of the present invention, the method further comprises the steps:
communicating information indicative of a physiological or an activity state of the patient to an external unit by the first implantable device, wherein the information indicative of a physiological or an activity state of the patient is measured via the first implantable device or the second implantable device.

The present invention particularly stations the hardware and algorithmic support needs for an LP (second implantable device) in a separate cardiac monitoring hub (first implantable device). Such allocation frees up the need for inclusion of such considerations in LPs installed within the heart volume thus better enabling them to optimize size and power management needs. Particularly, this approach leans on viable body-area network communication for an overall gain in system performance and does so without demanding that beat-by-beat implant-to-implant dialogues exist. Beyond supporting improved design restructuring, the system enables opportunities for aligning deep implants (e.g., the LP) with the remote monitoring infrastructure in an efficient way.

Further features, aspects, objects, advantages, and possible applications of the present invention will become apparent from a study of the exemplary embodiments and examples described below, in combination with the Figure, and the appended claims.

### DESCRIPTION OF THE DRAWINGS

Other advantages and expedient features of the present invention follow from the following description of sample embodiments, which make reference to the Figure. The Figure is as follows:

FIG. 1 shows an embodiment of an implantable device system according to the present invention.

### DETAILED DESCRIPTION

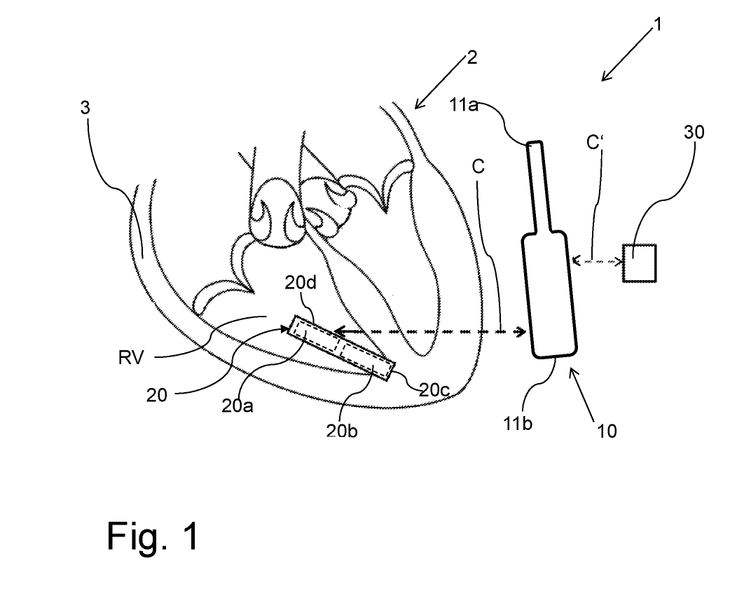

FIG. 1 shows an implantable device system 1 for applying electrical stimulation to a patient, particularly to the heart 2 of the patient, according to the present invention. The system 1 comprising: a first implantable device 10 configured to measure at least one parameter indicative of a physiological or activity state of the body of the patient, particularly of the heart 2 of the patient, and a second implantable device 20 configured to apply and/or adapt electrical stimulation to the patient/heart 2 for therapy purposes in response to said at least one parameter, wherein the first implantable device 10 is further configured to communicate said at least one parameter to the second implantable device 20.

Further, particularly, the second implantable device 20 is an implantable lead-less pacemaker (LP) 20 which is configured to apply electrical stimulation to the heart 2 of the patient in the form of electrical pacing pulses, particularly in order to provide anti-bradycardia pacing and/or anti-tachycardia pacing (ATP). Particularly, the LP 20 may comprise a hermetically sealed housing 20d enclosing a pulse generator 20b for generating said pacing pulses and a battery 20a for supplying energy to the pulse generator 20b. The lead-less pacemaker 20 may further comprise fastening means provided on the housing 20d for fastening the LP 20 to the chamber/ventricle (here the right ventricle RV), and a pacing electrode 20c provided, e.g., on an end of the housing 20d of the LP 20 for applying said electrical pacing pulses to the heart 2. Regarding the LP 20, the notion lead-less means that the primary electrode 20c of the LP 20 is not connected via an external lead to the housing 20d of the LP 20 but is provided on the housing 20d. Secondary electrodes (not shown in figure) may exist on lead like extensions from the housing 20d in some embodiments.

Furthermore, particularly, the first implantable device 10 is a cardiac monitoring device 10, particularly an implantable loop recorder 10 that is configured to receive electrocardiogram signals via a pair of electrodes 11a and 11b and to store the corresponding electrocardiogram (ECG) in a (e.g., circular) buffer of the recorder 10. Alternatively, the first implantable device 10 can be a subcutaneous implantable cardioverter-defibrillator that is configured to apply electrical stimulation to the heart (2) for providing defibrillation of the heart (2).

In either embodiment, the respective first implantable device 10 forms a subcutaneous cardiac monitoring hub 10 that particularly occupies a larger volume than that of the LP 20. This larger volume is particularly used to accommodate a higher capacity battery, added componentry, and greater processing capabilities as described herein. Further, because the hub 10 is implanted subcutaneously, it is easy to explant and replace when the battery runs low, whereas the LP 20 is much more difficult to explant. Further, because the hub 10 is preferably configured to reside near the patient's skin surface, it can gather system-affiliated data and send it to a remote monitoring infrastructure 30 without any need for direct patient interaction.

To minimize the overhead affiliated with this revised approach to baseline feature support, the coordination of message relay within the patient's body may also be aligned with prevailing cardiac activity to enable low-power receiver implementations within the LP 20. The triggered calls for action from the hub 10 could also be enabled as “loud” (e.g., High Energy Pulse (HEP)-like) signals that wake up the LP 20 without forcing the in-heart implant, i.e. the LP, to consume unfavorable amounts of energy waiting for such cues.

Particularly, the LP 20 is configured to apply electrical stimulation in the form of electrical pulses having a pulse rate to heart 2, wherein the system 1 is configured to adapt said electrical stimulation by adapting said pulse rate, wherein the hub 10 is configured to measure said at least one parameter indicative of an activity state of the patient, wherein the first implantable device is further configured to communicate a signal via a wireless communication link C to the LP 20 in case activity state measured by the hub 10 exceeds a pre-defined threshold, wherein in turn the LP is configured to adapt the pulse rate in response to said signal from the hub 10.

Communication link C is an ultra-low power communication link suitable for intrabody communication. In one embodiment, it is galvanic communication in which electrodes from one device put modulated current or voltage pulses into the body. These current or voltage pulses generate small E-fields in the body which are detected by electrodes of the second device. In an alternative embodiment, acoustic signals as, e.g., modulated ultrasonic signals are used for communication. Ultrasonic frequencies (>30 kHz) are used so that the patient is not bothered by hearing the signals. In both methods data is encoded in the modulation of the signals.

Furthermore, particularly, the hub 10 is configured to trigger a signal when the at least one measured parameter or another parameter measured by the hub 10 meets a pre-defined criterion that indicates the need for holter storage, wherein the signal upon reception by the LP transitions the hub 10 and the LP 20 into a dialogue mode, where the LP 20 streams data sensed by the LP (e.g., an ECG) to the hub 10 via a wireless communication link C for remote storage. The communication link C may support bi-directional communication. Particularly, storage of the ECG data may take place in the hub 10 or in a further external device 30,wherein the hub 10 may communicate with said external device 30 via a wireless communication link C′ (which may also support bi-directional communication). In some embodiments, the LP (20) can also initiate a holter storage where the LP streams data to the hub.

Such a setup would mean that information sensed within the heart 2 could be retained in the less volumetrically constrained hub 10. Further, coordinated snapshots would prove accessible wherein the hub 10 is configured to capture a sensed patient response of its own while also collecting the sensed data from the in-heart LP 20. By collecting two electrical signatures of patient behaviors assessed from different vantages within a shared survey window, it may prove possible to improve upon meaningful clinical diagnostic capabilities.

In another embodiment, the LP 20 contains enough memory to locally store a few ECG episodes. In this embodiment, the LP 20 can locally store a ECG episode and then later transmit it to the hub 10 for longer term storage. This embodiment eliminates the need for the ECG to be transmitted to the hub in real time, while still providing the benefit of not burdening the LP 20 with large amounts of memory for episode storage.

Particularly, according to a further embodiment, the hub 10 may be configured to conduct all remote monitoring interactions and particularly constitutes the only device of the system 1 according to the present invention that is capable of supporting direct interactions with an external unit 30. The external unit 30 may be a programming device for programming the LP 20 and/or the hub device 10, and for interrogating the LP, and further for performing follow-up tests on the LP, particularly via a wireless communication link C′. The external unit may also be a patient device where the data received from the hub device may be further processed, evaluated, displayed to users or the patient, and sent to other devices or a service center, or the like, for the purpose of remote monitoring of the physiological state of the patient. The external unit may also be an external service center where the data may be further processed, evaluated or the like, for the purpose of remote monitoring of the physiological state of the patient.

Particularly, according to an embodiment, the hub 10 and the LP 20 are configured to communicate all data that is to be transferred between the hub 10 and the LP 20 via a wireless body area network (WBAN).

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein.

Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

I/We claim:

1. An implantable device system for applying electrical stimulation to a patient, comprising:
a first implantable device configured to measure at least one parameter indicative of a physiological or an activity state of the patient,
a second implantable device configured to apply and/or adapt electrical stimulation to the patient in response to said at least one parameter, and
wherein the first implantable device is further configured to communicate information pertaining to said at least one parameter to the second implantable device.

2. The implantable device system according to claim 1, wherein the second implantable device is a pacemaker configured to apply electrical stimulation to a heart of the patient.

3. The implantable device system according to claim 2, wherein the second implantable device is an implantable lead-less pacemaker that is configured to be implanted into a chamber (RA, RV, LV) or on a chamber (RA, RV) of said heart of the patient.

4. The implantable device system according to claim 2, wherein the second implantable device is configured to apply electrical stimulation in the form of electrical pulses having a pulse rate to the patient, particularly to the heart of the patient, wherein the device system is configured to adapt said electrical stimulation by adapting said pulse rate, wherein the first implantable device is configured to measure said at least one parameter indicative of an activity state of the patient, wherein the first implantable device is further configured to communicate a signal (C) to the second implantable device in case said activity state exceeds one or more thresholds, wherein in turn the second implantable device is configured to adapt the pulse rate in response to said signal.

5. The implantable device system according to claim 1, wherein the first implantable device is configured to sense cardiac responses of the patient to the electrical stimulation of the second implantable device, wherein the cardiac response is in particular cardiac capture, wherein the first implantable device is configured to periodically assess the correspondence of said sensed cardiac responses of the patient to said electrical stimulation for grading its efficacy.

6. The implantable device system according to claim 1, wherein the first implantable device is configured to trigger a signal (C) when the at least one measured parameter meets one or more pre-defined criterion, wherein the signal upon reception by the second implantable device transitions the first and the second implantable device into a dialogue mode, where the second implantable device streams data sensed by the second implantable device to the first implantable device for storage.

7. The implantable device system according to claim 2, wherein the first implantable device is configured to detect electrocardiogram signals from the heart of the patient and is further configured to store said signals in a memory.

8. The implantable device system according to claim 2, wherein the first implantable device forms a subcutaneous implantable cardioverter-defibrillator (s-ICD) that is configured to apply electrical stimulation to the heart of the patient for providing defibrillation of the heart.

9. The implantable device system according to claim 1, wherein the first and the second implantable device are configured to communicate via a wireless body area network (WBAN).

10. The implantable device system according to claim 1, wherein the first implantable device is configured to perform data transfer with an external unit.

11. The implantable device system according to claim 10, wherein the external unit is a programmer device, a patient device, or hardware that interfaces with an external service center.

12. The implantable device system according to claim 1, wherein the first implantable device comprises at least one pair of electrodes for sensing electrical potentials.

13. A method for operating a device system according to claim 1, comprising the steps:
measuring at least one parameter indicative of a physiological or an activity state of the patient via a first implantable device,
communicating information pertaining to said at least one parameter from the first implantable device to a second implantable device,
applying and/or adapting electrical stimulation to the patient in response to said at least one parameter via the second implantable device.

14. The method for operating a device system according to claim 13, further comprising the steps:
communicating information indicative of a physiological or an activity state of the patient to an external unit by the first implantable device, wherein the information indicative of a physiological or an activity state of the patient is measured via the first implantable device or the second implantable device.
